(19) United States
(12) Patent Application Publication
(10) Pub. No.: US 2022/0160723 A1
(43) Pub. Date: May 26, 2022

(54) PHARMACOLOGICAL MITIGATION OF LATE-STAGE TOXEMIA

(71) Applicant: The Regents of the University of California, Oakland, CA (US)

(72) Inventors: Ethan Bier, La Jolla (YS); Prashant Jain, La Jolla, CA (US)

(21) Appl. No.: 17/602,700

(22) PCT Filed: Apr. 20, 2020
(86) PCT No.: PCT/US2020/028945

§ 371 Date: Oct. 8, 2021

Related U.S. Application Data
(60) Provisional application No. 62/835,597, filed on Apr. 18, 2019.

(51) Int. Cl. A61K 31/5377; A61K 31/506; A61K 31/44; A61K 31/277; A61P 39/02

(52) U.S. Cl. CPC A61K 31/5377; A61K 31/506; A61P 39/02; A61K 31/277; A61K 31/44

(57) ABSTRACT

Methods and compositions for treatment of cell-cell barrier associated conditions, such as caused by anthrax Edema toxin, by inhibiting receptor tyrosine kinase. The present invention provides methods of treating a cell-cell barrier disruption-associated condition in a subject comprising administering to a subject in need thereof a treatment effective amount of an inhibitor of insulin growth factor-1 receptor (IGF-1R) or IGF-1R down stream signaling.

### CROSS REFERENCE TO RELATED APPLICATIONS

This application claims the priority benefit of ITS. Provisional Application No. 62/835,597 filed Apr. 18, 2019, which application is incorporated herein by reference.

### GOVERNMENT SPONSORSHIP

This invention was made with government support under grant No. Ail 107 13 awarded by the National Institutes of Health. The government has certain rights in the invention.

### TECHNICAL FIELD

The present invention relates to pharmacological mitigation of fete-stage toxemia and cell-cell barrier disruptions.

### BACKGROUND

Anthrax disease, caused by Bacilluis anthracis, is a highly lethal infection with patient fatality rate between 45-85% during fulminant, toxemia-related late-stages of infection. Systemic release of anthrax Edema toxin (EX) during late-stage infection induces vascular collapse through endothelial barrier disruption, culminating in fatal hypovolemic shock, a hallmark of systemic anthrax infection. Existing therapeutic strategies to mitigate the effects of anthrax infections only target early-stage infection vis-a˜vis bacterial clearance (antibiotics} and toxin-host cell interactions (anti-toxin antibodies), but are ineffective in preventing toxemic-shock which is induced even after pathogen clearance. in fact, patients with fulminant infection require aggressive, continuous fluid drainage and assisted breathing, and no effective therapeutic interventions exist currently for this critical stage of infection.

Pathogen induced cell-cell barrier disruption (anthrax, cholera, traveler's diarrhea, gastroenteritis, pertussis, pneumonia) account for significant socio-economic impacts each year. Stand-alone antitoxin therapies such as those mentioned here can fulfill the unmet medical need for measures that significantly improve the survival rate of patients with severe infections, and lower the risk for development of antibiotic resistance.

Existing treatment regimens for anthrax include antibiotics such as Ciprofloxacin and Doxycyclin (60 day treatment) to eliminate the bacteria and antitoxin measures such as BioThrax (Anthrax Vaccine Adsorbed), Raxibacumab and Oblitoxaximab, which are recombinant vaccines against Bacillus anthracm Protective antigen (PA). However, these therapeutic measures offer limited protection, and only against early-stage infection. Specifically, while antibiotics reduce pathogen load, and rPA based anthrax vaccines prevent toxin interaction with host cells, therapeutic measures which target the fatality-related sequelae of cellular effects initiated following systemic release of toxins are thus far lacking.

High fatality rate of anthrax infections, despite intense antibiotic and supportive therapies, are primarily due to the continuing activities of anthrax exotoxins (FT and LT) released in the patient's circulatory system. Edema toxin or ET, a highly active adenylate cyclase that induces uncontrolled, pathological elevation in cellular levels of the second messenger cAMP (1) is a major virulence protein of Bacillus amhracis and mediates significant lethality during fulminant stages of infection (2). ET induces rapid disruption of the endothelial barrier, resulting in irreversible tissue damage and lethality due massive fluid loss resulting in cardiovascular collapse and hypovolemic shock. It is therefore imperative that new therapeutic measures be developed that effectively block the intracellular function of ET (i.e., cellular proteins/pathways co-opted to induce barrier instability), to reduce fatalities associated with anthrax toxemia.

### SUMMARY OF THE INVENTION

The present invention provides methods of treating a cell-cell barrier disruption-associated condition in a subject comprising administering to a subject in need thereof a treatment effective amount of an inhibitor of insulin growth factor-1 receptor (IGF-1R) or IGF-1R downstream signaling.

In embodiments, the present invention provides a method of treatment wherein the condition is caused by anthrax, cholera, traveler's diarrhea, gastroenteritis, pertussis, or pneumonia.

In embodiments, the present invention provides a method of treatment wherein the condition is caused by anthrax Edema toxin.
embodiments, the present invention provides a method of treatment wherein the inhibitor inhibits GTPase Rac1.
embodiments, the present invention provides a method of treatment wherein the inhibitor is NSC23766 inhibiting Rac1.
embodiments, the present invention provides a method of treatment wherein the inhibitor is a PI3K inhibitor or a MEK inhibitor,
in embodiments, the present invention provides a method of treatment wherein the inhibitor is GDC-0941 inhibiting PI3K.
In embodiments, the present invention provides a method of treatment wherein the inhibitor is AS703026 inhibiting MEK.

embodiments, the present invention provides a method of treatment wherein the inhibitor is AG1024 inhibiting IGF-1 R.

embodiments, the present invention provides a method of treatment wherein the condition is caused by chronic inflammation disorders, asthma, inflammatory bowel disease, atopic dermatitis, celiac disease, or Crohn's disease,

The present invention provides a method of treating a cell-cell barrier disruption in a subject comprising administering to a subject in need thereof a cell-cell barrier disruption inhibiting effective amount of an inhibitor of IGF-1R or IGF-1R downstream signaling.

In embodiments, the present invention provides a method of treating a cell. cell barrier wherein the condition is caused by anthrax. cholera, traveler's diarrhea, gastroenteritis, pertussis, or pneumonia.
embodiments, the present invention provides a method of treating a cell-cell barrier wherein the condition is caused by anthrax Edema toxin.

In embodiments, the present invention provides a method of treating a cell-eel! barrier wherein the inhibitor inhibits GTPase Rac1.

In embodiments, the present invention provides a method of treating a cell-cell barrier wherein the inhibitor is NSC23766 inhibiting Rad.

In embodiments, the present invention provides a method of treating a cell-cell barrier wherein the inhibitor is a PI3K inhibitor or a MBK inhibitor,

In embodiments, the present invention provides a method of treating a cell-cell barrier wherein the inhibitor is GDC-0941 inhibiting PI3K.

In embodiments, the present invention provides a method of treating a cell-cell barrier wherein the inhibitor is AS703026 inhibiting MEK.

In embodiments, the present invention provides a method of treating a cell-cell barrier wherein the inhibitor is AG1024 inhibiting IGF-1R.

### BRIEF DESCRIPTION OF THE DRAWINGS

FIGS. 1A-1B shows anthrax edema toxin induces cortical actin rearrangement in Human Brain Microvascular Endothelial cells (HBMEC).

FIGS. 2A-2E show ET induced actin rearrangement is dependent on Rac1 activation.

FIGS. 3A-3H show ET-mediated Rac1 activation and actin rearrangement is dependent on an early induction of IGF-1R, PI3K and MEK signaling.

FIGS. 4A-4I show ET induced IGF-1R, MEK and Rac1 signaling activate (dephosphorylate) the actin depolymerizing protein coftlin (CFL1).

FIG. 5 shows in vivo validation of barrier protective potential of MEK, PI3K, Rac1, Epac and IGF-1R inhibitors.

FIG. 6 shows a graphical model of ET-dependent barrier disruption.

### DETAILED DESCRIPTION

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the exemplary methods, devices, and materials are described herein.

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, 2^{nd} ed. (Sambrook et al, 1989); Oligonucleotide Synthesis (M. J. Gait, ed., 1984); Animal Cell Culture (R. I. Freshney, ed., 1987); Methods in Enzymology (Academic Press, Inc.); Current Protocols in Molecular Biology (F. M. Ausubel et al., eds., 1987, and periodic updates); PCR: The Polymerase Chain Reaction (Muilis et al, eds., 1994); Remington, The Science and Practice of Pharmacy, 20* ed., (Lippincott, Williams & Wilkins 2003), and Remington, The Science and Practice of Pharmacy, 22th ed., (Pharmaceutical Press and Philadelphia College of Pharmacy at University of the Sciences 2012).

The present invention provides methods of treating a cell-cell barrier disruption-associated condition in a subject comprising administering to a subject in need thereof a treatment effective amount of an inhibitor of insulin growth factor-1 receptor (IGF-1R) or IGF-1R downstream signaling.

In embodiments, the present invention provides a method of treatment wherein the condition is caused by anthrax, cholera, traveler's diarrhea, gastroenteritis, pertussis, or pneumonia.

In embodiments, the present invention provides a method of treatment wherein the condition is caused by anthrax Edema toxin.
in embodiments, the present invention provides a method of treatment wherein the inhibitor inhibits GTPase Rad.
in embodiments, the present invention provides a method of treatment wherein the inhibitor is NSC23766 inhibiting Rac1.

In embodiments, the present invention provides a method of treatment wherein the inhibitor is a PI3K inhibitor or a MEK inhibitor,

In embodiments, the present invention provides a method of treatment wherein the inhibitor is GDC-0941 inhibiting PI3K.

In embodiments, the present invention provides a method of treatment wherein the inhibitor is AS703026 inhibiting MEK.

In embodiments, the present invention provides a method of treatment wherein the inhibitor is AG1024 inhibiting IGF-1R

In embodiments, the present invention provides a method of treatment wherein the condition is caused by chronic inflammation disorders, asthma, inflammatory bowel disease, atopic dermatitis, celiac disease, or Crohn's disease.

The present invention provides a method of treating a cell-cell barrier disruption in a subject comprising administering to a subject in need thereof a cell-cell barrier disruption inhibiting effective amount of an inhibitor of IGF-1R or IGF-1R downstream signaling.

In embodiments, the present invention provides a method of treating a cell-cell barrier disruption wherein the disruption caused by anthrax, cholera, traveler's diarrhea, gastroenteritis, pertussis, or pneumonia.

in embodiments, the present invention provides a method of treating a cell-cd! barrier disruption wherein the condition is caused by anthrax Edema toxin.

In embodiments, the present invention provides a method of treating a cell-cell barrier disruption wherein the inhibitor inhibits GTPase Rac1.

In embodiments, the present invention provides a method o f treating a cell-cell barrier disruption wherein the inhibitor is NSC23766 inhibiting Rac1.

In embodiments, the present invention provides a method of treating a cell-cell barrier wherein the inhibitor is a P13K inhibitor or a MBK inhibitor,

In embodiments, the present invention provides a method of treating a cell-cell harrier disruption wherein the inhibitor is GDC-0941 inhibiting PI3K.

In embodiments, the present invention provides a method of treating a cell-cell barrier disruption wherein the inhibitor is A S703026 inhibiting MEK.

In embodiments, the present invention provides a method of treating a cell-cell barrier disruption wherein the inhibitor is AG1024 inhibiting IGF-1R.

In embodiments, the present invention provides a method of treating a cell-cell barrier disruption wherein the inhibitor is ESI09 inhibiting Epac.

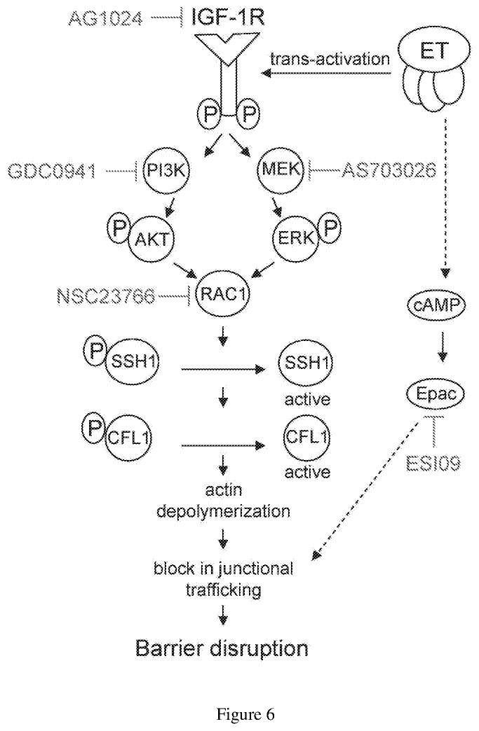

FIG. 6 shows a graphical model of ET-dependent barrier disruption where IGF-1R or IGF-1R downstream signaling can be inhibited in the methods of the present invention. Examples of therapeutic agents for use in the present invention include inhibitors of IGF-1R such as tyrphostins AG1024 (2-[(3-bromo-5˜iert-butyl-4-hydroxyphenyl)methylidene]propanedinitile). AG538, PyrroIo(2,3˜d)-pyrimidine derivatives such as NVP-AEW541, monoclonal antibodies such as figitumumab, and inhibiting RNA. Inhibitors of Rac1 activation include NSC23766 ((N(6)˜[2-[[4˜(diethylamino)-1-methylbutyl]amino]-6-methyl-4-pyrimidinyl]-2-methyl-4,6-quinolinediamine trihydrochloride), monoclonal antibodies, and inhibiting RNA. Phosphoinositide 3-kinase inhibitors (PI3K inhibitors) include GDC-0941 (pictilisib), monoclonal antibodies, and inhibiting RNA. MEK inhibitors include AS703026, GSK1120212 (trametinib), XL518 (cobimetinib), MEK162 (binimetinib), monoclonal antibodies, and inhibiting RNA. inhibitors of exchange factor directly activated by cAMP I (BPAC1) include ES109, monoclonal antibodies, and inhibiting RNA. IGF-1R or IGF-1R downstream signaling inhibitors are described and well-known in the art,

As used herein, the terms “comprises,” “comprising,” “includes,” “including,” “has,” “having,” “contains”, “containing,” “characterized by,” or any other variation thereof, are intended to encompass a non-exclusive inclusion, subject to any limitation explicitly indicated otherwise, of the recited components. For example, a fusion protein, a pharmaceutical composition, and/or a method that “comprises” a list of elements (e.g., components, features, or steps) is not necessarily limited to only those elements (or components or steps), but may include other elements (or components or steps) not expressly listed or inherent to the fusion protein, pharmaceutical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a fusion protein, pharmaceutical composition, and/or method that includes materials, steps, features, components, or elements, in addition to those literally disclosed, provided that these additional materials, steps, features, components, or elements do not materially affect the basic and novel characteristic(s) of the claimed invention. The term “consisting essentially of * occupies a middle ground between “comprising” and “consisting of’.

When introducing elements of the present invention or the preferred embodiment(s) thereof the articles “a”, “an”, “the” and “said” are intended to mean that there are one or more of the elements. The terms “comprising”, “including” and “having” are intended to be inclusive and mean that there may he additional elements other than the listed elements.

The term “and/or” when used in a list of two or more items, means that any one of the listed items can be employed by itself or in combination with any one or mom of the listed items. For example, the expression “A and/or B” is intended to mean either or both of A and B. i.e. A alone, B alone or A and B in combination. The expression “A, B and/or C” is intended to mean A alone, B alone, C alone, A and B in combination, A and C in combination, B and C in combination or A, B, and C in combination.

It is understood that aspects and embodiments of the invention described herein include “consisting” and/or “consisting essentially of” aspects and embodiments.

It should be understood that the description in range format is merely for convenience and brevity and should not be construed as an inflexible limitation on the scope of the invention. Accordingly, the description of a range should be considered to have specifically disclosed all the possible sub-ranges as well as individual numerical is values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc,, as well as individual numbers within that range, for example, f, 2, 3, 4, 5, and 6. This applies regardless of the breadth of the range. Values or ranges may be also be expressed herein as “about,” from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself in embodiments, “about” can be used to mean, for example, within 10% of the recited value, within 5% of the recited value, or within 2% of the recited value.

As used herein, “patient” or “subject” means a human or animal subject to be treated.

As used herein the terra “pharmaceutical composition” refers to a pharmaceutical acceptable compositions, wherein the composition comprises a pharmaceutically active agent, and in some embodiments further comprises a pharmaceutically acceptable carrier. In some embodiments, the pharmaceutical composition may be a combination of pharmaceutically active agents and carriers.

The term “combination” refers to either a fixed combination in one dosage unit form, or a kit of parts for the combined administration where one or more active compounds and a combination partner (e.g., another drug as explained below, also referred to as “therapeutic agent” or “co-agent”) may be administered independently at the same time or separately within time intervals. In some circumstances, the combination partners show a cooperative, e.g., synergistic effect. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g.. a compound and a combination partner, are both administered to a patient simultaneously in the form of a single entity or dosage. The term “non-fixed combination” means that the active ingredients, e.g., a compound and a combination partner, are both administered to a patient as separate entities either simultaneously, concurrently or sequentially with no specific time limits, wherein such administration provides therapeutically effective levels of the two compounds in the body of the patient. The later also applies to cocktail therapy, e.g., the administration of three or more active ingredients,

Preferred routes the administration, such as oral, buccal, nasal, peritoneal, inhalation, ocular, optic, rectal, vaginal, cutaneous and transdermal, preferred formulations, such as liquids, capsules, tablets, and preferred dosages can be routinely determined based upon the disease or cell-cell barrier disruption, the condition of the subject, and judgement of the attending physician.

As used herein the term “pharmaceutically acceptable” means approved by a regulatory agency of the Federal or a state government or listed in the U.S. Pharmacopoeia, other generally recognized pharmacopoeia in addition to other formulations that are safe for use in animals, and more particularly in humans and/or non-human mammals.

As used herein the term “pharmaceutically acceptable carrier” refers to an excipient, diluent, preservative, solubilizer, emulsifier, adjuvant, and/or vehicle with which dernethylation compound(s), is administered. Such carriers may be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose may also be a carrier. Methods for producing compositions in combination with carriers are known to those of skill in the art. In some embodiments, the language “pharmaceutically acceptable carrier” is intended to include any and all solvents, dispersion media, coatings, isotonic and absorption delaying agents, and the like, compatible with pharmaceutical administration. The use of such media and agents for pharmaceutically active substances is well known in the art. See, e.g., Remington, The Science and Practice of Pharmacy, 20th ed., (Lippincott, Williams & Wilkins 2003). Except insofar as any conventional media or agent is incompatible with the active compound, such use in the compositions is contemplated.

As used herein, “therapeutically effective” refers to an amount of a pharmaceutically active compound(s) that is sufficient to treat or ameliorate, or in some manner reduce the symptoms associated with diseases and medical conditions. When used with reference to a method, the method is sufficiently effective to treat or ameliorate, or in some manner reduce the symptoms associated with diseases or conditions. For example, an effective amount in reference to conditions or diseases is that amount which is sufficient to block or prevent onset; or if disease pathology has begun, to palliate, ameliorate, stabilize, reverse or slow progression of the disease, or otherwise reduce pathological consequences of the disease. In any case, an effective amount may be given in single or divided doses,

As used herein, the terms “treat;” “treatment,” or “treating” embraces at least an amelioration of the symptoms associated with diseases in the patient, where amelioration is used in a broad sense to refer to at least a reduction in the magnitude of a parameter, e.g. a symptom associated with the disease or condition being treated. As such, “treatment” also includes situations where the disease, disorder, or pathological condition, or at least symptoms associated therewith, are completely inhibited (e.g. prevented from happening) or stopped (e.g. terminated) such that the patient no longer suffers from the condition, or at least the symptoms that characterize the condition.

As used herein, and unless otherwise specified, the terms “prevent” “preventing” and “prevention” refer to the prevention of the onset, recurrence or spread of a disease or disorder, or of one or more symptoms thee of in certain embodiments, the terms refer to the treatment with or administration of a compound or dosage form provided herein, with or without one or more other additional active agent(s), prior to the onset of symptoms, particularly to subjects at risk of disease or disorders provided herein. The terms encompass the inhibition or reduction of a symptom of the particular disease. In certain embodiments, subjects with familial history of a disease are potential candidates for preventive regimens. In certain embodiments, subjects who have a history of recurring symptoms are also potential candidates for prevention. In this regard, the term “prevention” may be interchangeably used with the term “prophylactic treatment.” In embodiments herein, the methods for treatment are applicable for methods of prevention.

As used herein, and unless otherwise specified, a “prophylactically effective amount” of a compound is an amount sufficient to prevent a disease or disorder, or prevent its recurrence. A prophylactically effective amount of a compound means an amount of therapeutic agent, alone or in combination with one or more other agent(s), which provides a prophylactic benefit in the prevention of the disease. The term “prophylactically effective amount” can encompass an amount that improves overall prophylaxis or enhances the prophylactic efficacy of another prophylactic agent.

As used herein, and unless otherwise specified, a compound described herein is intended to encompass all possible stereoisomers, unless a particular stereochemistry is specified. Where structural isomers of a compound are interconvertible via a low energy barrier, the compound may exist as a single tautomer or a mixture of tautomers. This can take the form of proton tautomerism; or so-called valence iautonierism in the compound, e.g., that contain an aromatic moiety.

The term “antibody” as used herein encompasses monoclonal antibodies (including full length monoclonal antibodies), polyclonal antibodies, multi-specific antibodies (e.g., bi-specific antibodies), and antibody fragments so long as they exhibit the desired biological activity of binding to a target antigenic site and its isofonns of interest.

The term “antibody fragments” comprise a portion of a full length antibody, generally the antigen binding or variable region thereof. The term “antibody” as used herein encompasses any antibodies derived from any species and resources, including but not limited to, human antibody, rat antibody, mouse antibody, rabbit antibody, and so on, and can be synthetically made or naturally-occurring.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The “monoclonal antibodies” may also be isolated from phage antibody libraries using the techniques known in the at.

The monoclonal antibodies herein include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. As used herein, a “chimeric protein” or “fusion protein” comprises a first polypeptide operatively linked to a second polypeptide. Chimeric proteins may optionally comprise a third, fourth or f f h or other polypeptide operatively linked to a first or second polypeptide. Chimeric proteins may comprise two or more different polypeptides. Chimeric proteins may comprise multiple copies of the same polypeptide. Chimeric proteins may also comprise one or more mutations in one or more of the polypeptides. Methods for making chimeric proteins are well known in the art.

The invention includes the identification of three exemplary small molecule inhibitors of barrier disrupting toxins (NSC23766, GDC-0941 and AS703026). Using anthrax Edema toxin-induced cell-cell barrier dysfunction as a model system, pharmacological inhibitors, including clinically safe drugs, which can serve as novel antitoxin measures for ameliorating late-stage, and potentially, fatality-related effects of ET-toxemia were identified. Utilizing biochemical and cell-based approaches, an unknown mechanism was discovered wherein Edema toxin (ET) induces significant remodeling of the actin network following rapid activation of Insulin Growth Factor-1R (IGF-1R) signaling, ultimately resulting in cell-cell barrier disruption. Pharmacological intervention of IGF-I R signaling mediators, including inhibitors against IGF-1R (AG1024), Rac.1 (NSC23766) and clinically approved compounds such as P13K (GDC-0941) and MEK (AS703026), strongly inhibit ET-mediated actin remodeling in cultured endothelial cells (HBMEC) and more importantly, provide near complete protection against El-induced footpad swelling (edema) in an in vivo mouse model. The invention contemplates the use of these and other known compounds, and those discovered in the future, to inhibit at various points IGF-1R signaling in ET-mediated barrier disruption in a subject in need.

The invention discloses the critical involvement of Receptor Tyrosine Kinase (IGF-1R) signaling in ET-mediated barrier disruption, and the discovery of pharmacological inhibitors to ameliorate the organismal consequences of ET-toxemia.

Therapeutic measures effectively targeting late-stage ET-toxemia are a critical unmet medical need in the treatment of anthrax infections, importantly, in vivo evidence in mice is provided supporting the efficacy of late-stage intervention, including those with clinically approved PI3K and MEK inhibitors, in combating ET-toxemia.

Previously, the role of a cAMP effector Epac, in the deregulation of Rabl 1 GTPase mediated exocyst vesicle trafficking of proteins to cell-cell adherens junction, thereby resulting in barrier disruption (3) was described. In the present invention, cell-based and biochemical investigations in a relevant human endothelial cell line HBMEC, reveal that ET-mediated barrier disruption is strongly linked to early, and robust changes in the cortical actin arrangement which occurs within 4 h of ET treatment. Cellular actin network, due to their function as anchors for junctional protein complexes (4), and vesicle transport (5) are indispensable for barrier maintenance. In-depth mechanistic characterization of ET-dependent cellular effects reveal a critical role of the cell surface Insulin Growth Factor-1 receptor (IGF-1R), a Receptor Tyrosine Kinase (RTK) family protein, in ET induced actin rearrangement and endothelial barrier disruption. Significantly, this invention shows that pharmacological inhibition of IGF-1R with AG1024, or it's downstream effectors PI3K, MEK and Rack with GDC-0941 , AS703026 and NSC23766, respectively almost completely rescue ET-mediated actin rearrangement and cell-cell barrier disruption in the cultured HBMEC model. More importantly, the therapeutic potential of these drugs were validated in vivo in a mouse footpad swelling assay, where mice pre-treated with each of these drugs showed almost complete protection against ET-induced footpad edema. In vivo proof-of-concept data in mice demonstrates the human therapeutic potential of these inhibitors in anthrax ET-induced edema,

This is the first report of a bacterial toxin co-opting the IGF-1R signaling mediators to promote barrier disruption such as cholera, traveler's diarrhea, gastroenteritis, pertussis, pneumonia, etc. The therapeutic compounds discovered here, which include but is not limited to clinically approved PI3K and MEK inhibitors, provides a novel set of intervention strategies to counter late-stage, toxemia-related symptoms in subjects with these disorders.

This invention has uncovered a novel role of IGF-1 R/PI3K/MEK and Rac1 signaling in anthrax ET-mediated barrier disruption. In vivo proof-of-concept data demonstrates the effectiveness of this therapeutic approach in blocking the organismal consequences of ET-intoxication. The commercial applications of the invention, however, go well beyond applications in treating patients with late-stage anthrax infections. Of note, barrier disruption is the underlying cause of several debilitating and final pathologies associated with infectious diseases such as cholera, pertussis (whooping cough), traveler's diarrhea etc., and also for chrome inflammatory disorders such as asthma, atopic dermatitis, celiac disease and Crohn's disease. These maladies amount to a staggering socio-economic burden worldwide and are within the focus areas of major biotechnology and pharmaceutical firms in US and elsewhere. Specifically, multiple therapeutic targets central to this approach (PB K/AKT, MEK/ERK, Rac1) are emerging as key components of pathogenesis in asthma (6-8), inflammatory bowel disease (9) and atopic Dermatitis (10).

### EXAMPLES

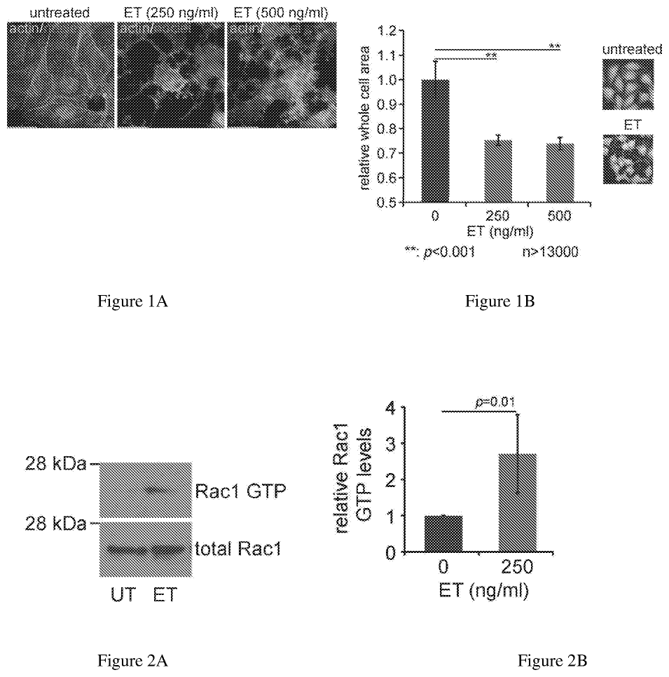

FIGS. 1A-1B show anthrax edema toxin induces cortical actin rearrangement in Human Brain Microvascular Endothelial cells (HBMEC). FIG. 1A shows ET induces significant rearrangement (clustering) of cortical actin network in HBMEC. Cells were treated with 250 and 500 ng/ml ET for 4 h and stained with Aiexa 488 conjugated phalloidin to visualize F-actiu. FIG. 1B shows medium throughput quantification of ‘whole cell area’ of HBMEC that were treated with ET for 4 h, fixed and stained with CellMask green to visualize cytoplasm. Cell area was quantified from 12 wells for each treatment (n-number of cells) using automated image analysis software. ET induced significant reduction in whole cell area (inset-representative picture). p value indicates standard deviation.

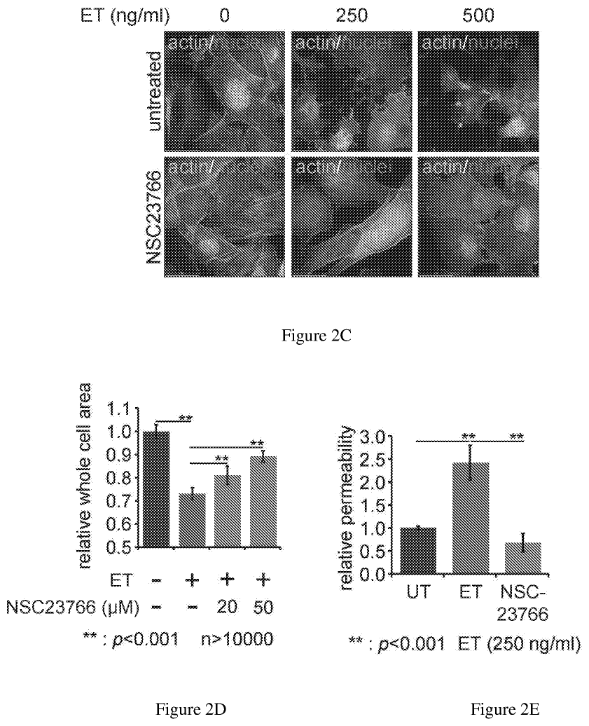

FIGS. 2A-2E show ET induced actin rearrangement is dependent on Rac1 activation. FIG. 2A shows HB MEC were treated with 250 ng/ml ET for 4 h. Whole cell lysates were incubated with PAK-Beads, which specifically bind to GTP-bound Rac1. Bead bound Rac1-GTP and total Rac1 levels in cell lysate were determined by western blot with anti-Rac1 antibody. ET induced significant increase in Rac1-GTP (i.e. active Rac1) levels (see FIG. 2B). FIG. 2B shows levels of Rac1-GTP relative to total Rac1 quantified from 2 independent experiments, p value indicates standard deviation. FIG. 2C shows HBMEC were treated with 250 and 500 ng/ml ET for 4 h, in the presence or absence of Rac inhibitor NSC23766 and stained with Alexa 488 conjugated phalloidin to visualize F-actin. Rac1 inhibition almost completely inhibited ET induced cortical actin rearrangement. FIG. 2D shows medium throughput quantification of ‘whole cell area’ of HBMEC, treated with ET (250 ng/ml) for 4 h, in the presence or absence of Rad inhibitor NSC23766 at indicated concentrations. Cells were fixed and stained with cell mask green to visualize cytoplasm. Cell area was quantified from 8 wells for each treatment («-number of cells) using automated image analysis software. Rac1 inhibition significantly reduced ET induced cell area reduction in an inhibitor dose dependent manner. p value indicates standard deviation. FIG. 2E shows ET induces the disruption of HBMEC monolayer integrity. MRMEC were grown to confluence in transwell inserts, and treated with ET (250 ng/ml) for 24h, in the presence or absence of Rac1 inhibitor NSC23766 (50 μM). Changes in monolayer permeability were assessed by assaying the diffusion of Evans blue dye from apical to basal chamber of transwell inserts. Rac1 inhibition significantly reduced ET induced disruption of monolayer integrity. Data represent mean changes in monolayer permeability from 2 independent experiments. p value indicates standard deviation.

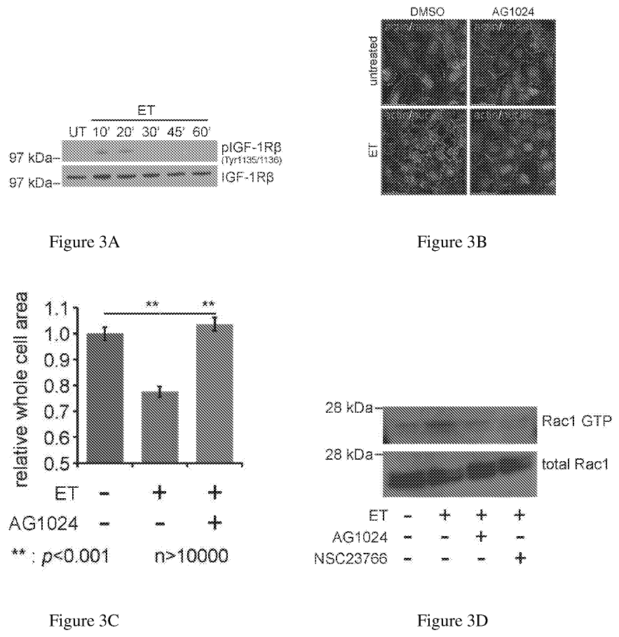

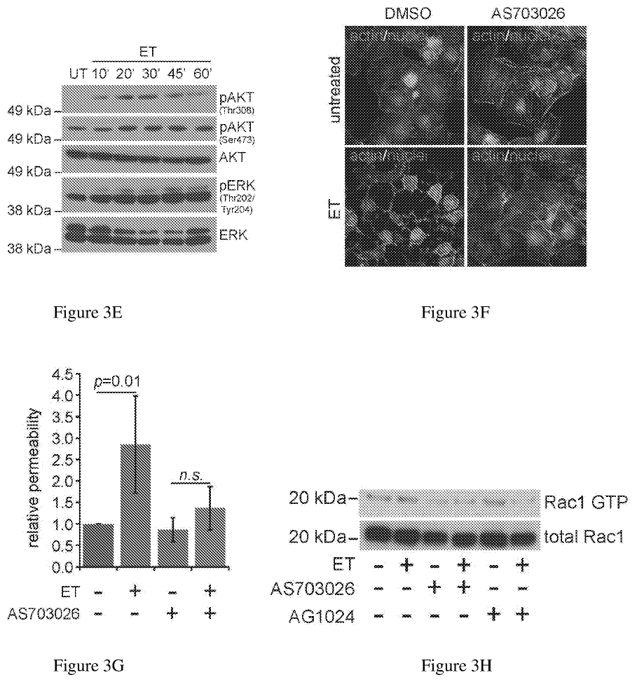

FIGS. 3A-3H show ET-mediated Rac1 activation and actin rearrangement is dependent on an early induction of IGF-1R, PI3K and MEK signaling. FIG. 3A shows HBMEC were treated with 250 ng/ml ET for indicated time periods. Activation of IGF-1R in ET˜ireated and control cells was assessed by monitoring the levels of IGF-1R phosphorylated at Tyrosine 1135 and 1136 relative to total IGF-1R in whole cell lysates by western blot. ET induced a rapid (within 10 minutes), but transient activation of IGF-1R. FIG. 3B shows HBMEC were treated with 250 ng/ml ET for 4 h, in the presence or absence of IGF-1R inhibitor AG 1024 and stained with Alexa 568 conjugated phalloidin to visualize F-actin. IGF-1R inhibition almost completely inhibited ET induced cortical actin rearrangement. FIG. 3C shows medium throughput quantification of ‘whole cell area’ of HBMEC, treated with ET (250 ng/ml) for 4 h, in the presence or absence of IGF-1R inhibitor AG 1024. Cells were fixed and stained with CellMask green to visualize cytoplasm. Cell area was quantified from 8 wells for each treatment (n-number of cells) using automated image analysis software. IGF-I R inhibition completely .rescued ET induced cell area reduction. p value indicates standard deviation. FIG. 3D shows HBMEC were treated with 250 ng/ml ET for 4 h, in the presence or absence of IGF-1R inhibitor AG 1024 and Rac1 inhibitor NSC23766. Whole cell lysates were incubated with PAK-Beads, which specifically bind to GTP-bound Rac1 . Bead bound Rac1-GTP and total Rac1 levels in cell lysate were determined by western blot with anti-Rac 1 antibody. IGF-1R and Rac1 inhibition completely rescued ET induced Rac1 activation. FIG. 3E shows HBMEC were treated with 250 ng/ml ET for indicated time periods. Total and phosphorylated 1GF-1R (Thr 308 and Ser 473), as well as those of ERK (Thr 202 and Tyr 204) was determined by western blot. ET induced a rapid (within 10-20 minutes) phosphorylation of AKT and ERK. FIG. 3F shows HBMEC were treated with 250 ng/ml ET for 4 h, in the presence or absence of MEK inhibitor AS703026 and stained with Alexa 488 conjugated phalloidin to visualize F-actin. MEK (upstream to ERK) inhibition significantly reduced ET induced actin rearrangement. FIG. 3G shows HBMEC were grown to confluence in transwell inserts, and treated with ET (250 ng/ml) for 24 h, in the presence or absence of MEK inhibitor AS703026 (50 μM). Changes in monolayer to permeability were assessed by assaying the diffusion of Evans blue dye from apical to basal chamber of transwell. MEK inhibition significantly reduced ET induced disruption of monolayer integrity. Data represent mean changes in monolayer permeability from 2 independent experiments. p value indicates standard deviation. FIG. 3H shows HBMEC were treated with 250 ng/ml ET tor 4 h, in the presence or absence of IGF-1R inhibitor AG1024 and MEK inhibitor 4 S703026. Whole cell lysates were incubated with PAK-Beads, which specifically bind to GTP-hound RacL Bead bound Rac1-GTP and total Rac1 levels in cell lysate were determined by western blot with anti-Rac1 antibody. IGF-1R and MEK inhibition significantly inhibited ET induced Rac1 activation.

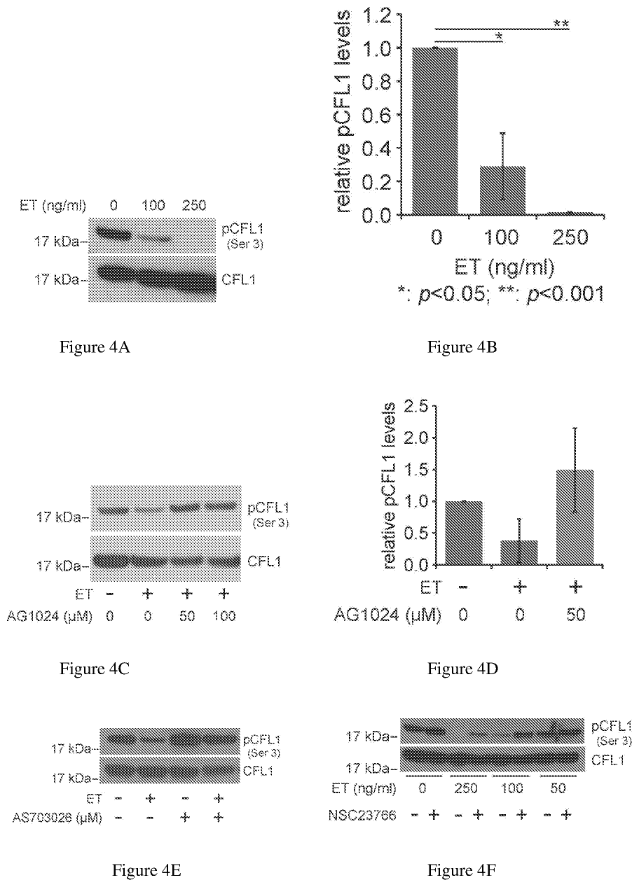

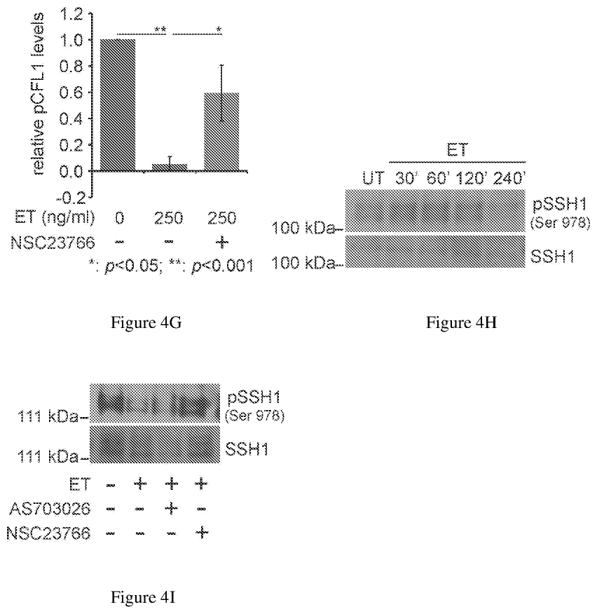

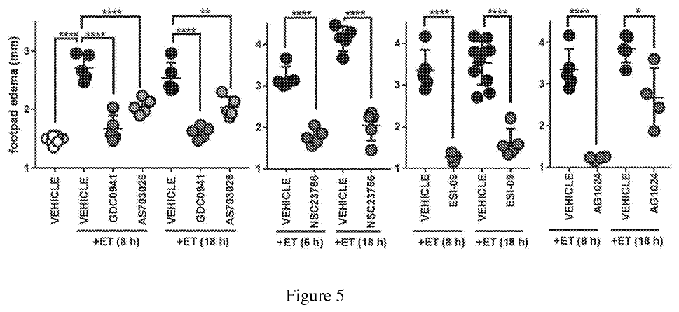

FIGS. 4A-4I show ET induced IGF-1R, MEK and Rac1 signaling activate (dephosphorylate) the actin depolymerizing protein coflin (CFL1). FIG. 4A shows HBMEC were treated with 100 and 250 ng/ml ET for 4 h, Total and phosphorylated CPU (Serine 3) levels were determined by western blot. ET induced a significant decrease in phosphorylated CFL1. (see FIG. 4B). FIG. 4B show levels of phosphorylated CFL1 relative to total CFL1 quantified from 2 independent experiments. p values indicate standard deviation. FIG. 4C shows HBMEC were treated with 250 ng/ml ET for 4 h, in the presence or absence of indicated concentrations of the IGF-1R inhibitor AG1024. Total and phosphorylated CFL1 (Serine 3) levels were determined by western blot. IGF-1R inhibition significantly inhibited ET induced CFL1 activation. (see FIG. 4D). FIG. 4D show levels of phosphorylated CFL1 relative to total CFL1 in whole cell lysates from HBMEC treated with ET in the presence or absence of IGF-1R inhibitor AG1024 and quantified from 2 independent experiments. FIG. 4E shows HBMEC were treated with 250 ng/ml ET for 4 h, in the presence or absence of MEK inhibitor AS703026. Total and phosphorylated CFL1 (Serine 3) levels were determined by western blot. MEK inhibition significantly inhibited ET induced CFL1 activation. FIG. 4F shows HBMEC were treated with 50, 100 and 250 ng/nil ET for 4 h, in the presence or absence of the Rac1 inhibitor NSC23766. Total and phosphorylated CFL1 (Serine 3) levels were determined by western blot. Rac1 inhibition significantly inhibited ET induced CFL1 activation. (see FIG. 4G). FIG. 4G show levels of phosphorylated CFL1 relative to total CFL1 in whole cell lysates from HBMEC treated with ET in the presence or absence of the Rac1 inhibitor NSC23766 and quantified from 2 independent experiments. p values indicate standard deviation. FIG. 4H shows HBMEC were treated with 250 ng/ml ET for indicated time periods. Activation (dephosphorylation) of SSH1 in ET-treated and control cells was assessed by monitoring the levels of SSHI phosphorylated at Serine 978 relative to total SSHI in whole cell lysates by western blot. SSHI phosphorylation was reduced relative to untreated control cells at 240 min after ET treatment. FIG. 4I shows HBMEC were treated with 250 ng/ml ET for 4 h, in the presence or absence of the MEK inhibitor AS703026 and Rac1 inhibitor NSC23766. Total and phosphorylated CFL1 (Serine 3) levels were determined by western blot. Rac1 inhibition significantly inhibited ET induced CFL1 activation.

FIG. 5 shows in vivo validation of barrier protective potential of MEK, PI3K, Rac1, Epac and 1GE-1R inhibitors. Pre-treatment of mice with MEK inhibitor AS703026 (33 mg/kg), PI3K inhibitor GDC0941 (75 mg/kg), Rad inhibitor NSC23766 (10 mg/kg), Epac inhibitor ES1-09 (10 mg/kg) or 1GF-1R inhibitor AG1024 (10 mg/kg) consistently block footpad swelling (edema) measured at indicated time periods following ET (0.15 μg ET/paw) injection. p values indicate standard deviation (****p<0.0001, **p<0.01, *p<0.05).

FIG. 6 shows a graphical model of ET-dependent barrier disruption. ET induces transactvation of IGF-1R, resulting in the activation of the downstream effectors, FI3K and MEK, and their respective substrates AKT and ERK. AKT and ERK signaling is required for the activation of Rac1, which activates the phosphatase SSHI to dephosphorylate, and thence activate CFL1. The dephosphorylation of actin by CFL1 weakens cell-cell barrier, possibly by blocking junctional trafficking in addition to the block initiated by the cAMP effector Epac, or directly via the loss of anchoring at the sites of interaction between] unction stabilizing proteins. Pharmacological inhibition of IGF-1R, PBK, MEK, Rac2 and Epac (inhibitors are indicated in red font) prevent ET-mediated barrier destabilization.

### REFERENCES

1. Leppla S B. Anthrax toxin edema factor; a bacterial adenylate cyclase that increases cyclic AMP concentrations of eukaryotic cells. Proceedings of the National Academy of Sciences of the United States of America. 1982;79(10):3162-6. Epub May 1, 1982.

2. Firoved A M, Miller G F, Moayerl M, Kakkar R, Shen Y, Wiggins J F, et al. Bacillus anthracis edema toxin causes extensive tissue lesions and rapid lethality in mice. The American journal of pathology. 2005; 167(5): 1309-20. Epub Oct. 28, 2005.

3. Guichard A, Jain P, Moayerl M, Schwartz R, Chin S, Zhu L, et al. Anthrax edema toxin disrupts distinct steps in. Rabl 1-dependent junctional transport. PLoS pathogens. 2017;13(9):e1006603 Epub Sep. 26, 2017.

4. Hartsock A, Nelson W J. Adherens and tight junctions: structure, function and connections to the actio cytoskeleton. Biochimica et biophysica acta. 2008; 177S(3):660-9. Epub Sep. 15, 2007.

5. Colonne P M, Wine-hell C G, Voth D E. Hijacking Host Cell Highways: Manipulation of the Host Actin Cytoskeleton by Obligate Intracellular Bacterial Pathogens. Frontiers in cellular and infection microbiology. 2016;6:107. Epub Oct. 8, 2016.

6. Wagh A D, Sharma M, Mahapatra J, Chatterjee A, Jain M, Addepalli V.

Investigation into the Role of PBK and JAK3 Kinase Inhibitors in Murine Models of Asthma. Frontiers in pharmacology. 2017;8:82. Epub Mar. 16, 2017.

7. Ito K, Caramon G, Adcock I M. Therapeutic potential of phosphatidylmositol 3-kinase inhibitors in inflammatory' respiratory disease. The Journal of pharmacology and experimental therapeutics. 2007;321(1):1-8, Epub Oct. 6, 2006.

8. Ghigo A, Damilano F, Braccini L, Hirsch E. PI3K inhibition in inflammation: Toward tailored therapies for specific diseases. BioEssays: news and reviews in molecular, cellular and developmental biology. 20 10;32(3):185-96. Epub Feb. 8, 2010.

9. Tokuhira N, Kitagishi Y, Suzuki M, Minami A, Nakanishi A, Ono Y, et al. PI3K/AKT/PTEN pathway as a target for Crohn's disease therapy (Review). international journal of molecular medicine. 2015;35(1); 10-6, Epub Oct. 30, 2014.

10. Stark A K, Sriskantharajah S, Hessel E M, Okkenhaug K. PI3K inhibitors in inflammation, autoimmunity and cancer. Current opinion in pharmacology. 2015;23:82-91. Epub Jun. 21, 2015.

1. A method of treating a cell-cell barrier disruption-associated condition in a subject comprising administering to a subject in need thereof a treatment effective amount of an inhibitor of insulin growth factor-1 receptor (IGF-1R) or IGF-1R downstream signaling.

2. The method of claim 1, wherein the condition is caused by anthrax, cholera, traveler's diarrhea, gastroenteritis, pertussis, or pneumonia.

3. The method of claim 1, wherein the condition is caused by anthrax Edema toxin.

4. The method of claim 1, wherein the inhibitor inhibits GTPase Rac1.

5. The method of claim 4, wherein the inhibitor is NSC23766 inhibiting Rac1.

6. The method of claim 1, wherein the inhibitor is a PI3K inhibitor or a MEK inhibitor.

7. The method of claim 6, wherein the inhibitor is GDC-0941 inhibiting PI3K.

8. The method of claim 1, wherein the inhibitor is AS703026 inhibiting MEK.

9. The method of claim 1, wherein the inhibitor is AG1024 inhibiting IGF-1R.

10. The method of claim 1, wherein the condition is caused by chronic inflammation disorders, asthma, inflammatory bowel disease, atopic dermatitis, celiac disease, or Crohn's disease.

11. A method of inhibiting a cell-cell barrier disruption in a subject comprising administering to subject in need thereof a cell-cell barrier disruption inhibiting effective amount of an inhibitor of IGF-1R or IGF-1R downstream signaling.

12. The method of claim 11, wherein the disruption is caused by anthrax, cholera, traveler's diarrhea, gastroenteritis, pertussis, or pneumonia.

13. The method of claim 11, wherein the disruption is caused by anthrax Edema toxin.

14. The method of claim 11, wherein the inhibitor inhibits GTPase Rac1.

15. The method of claim 14, wherein the inhibitor is NSC23766 inhibiting Rac1.

16. The method of claim 11, wherein the inhibitor is a PI3K inhibitor or a MEK inhibitor.

17. The method of claim 16, wherein the inhibitor is GDC-0941 inhibiting PI3K.

18. The method of claim 16, wherein the inhibitor is AS703026 inhibiting MEK.

19. The method of claim 11, wherein the inhibitor is AG1024 inhibiting IGF-1R.

20. The method of claim 11, wherein the subject is a human.
